(19) United States
(12) Patent Application Publication
(10) Pub. No.: US 2024/0115884 A1
(43) Pub. Date: Apr. 11, 2024

(54) ACCESSORY FOR A RADIOTHERAPY DEVICE

(71) Applicant: Elekta limited, Crawley (GB)

(72) Inventors: Michael Richard Healey, Crawley (GB); Amit Kumar Ravi, Crawley (GB)

(21) Appl. No.: 17/905,600

(22) PCT Filed: Mar. 3, 2021
(86) PCT No.: PCT/EP2021/055402

§ 371 Date: Sep. 2, 2022

(30) Foreign Application Priority Data
Mar. 4, 2020 (GB) 2003145.6

(51) Int. Cl. A61N 5/10

(52) U.S. Cl. CPC A61N 5/1075; A61N 5/1065; A61N 2005/1089; A61N 2005/1095

(57) ABSTRACT

An accessory for a radiotherapy device comprises a first electrical connector for forming an electrical connection to a radiotherapy device, and a signal emitter. The signal emitter is configured to emit a signal in order for the accessory to emulate the connection of a second accessory, comprising a beam control apparatus, to the radiotherapy device.

This application relates to an accessory for a radiotherapy device.

### BACKGROUND

Radiotherapy (or ‘therapeutic radiotherapy’) can be described as the use of ionising radiation to treat a human or animal body. Radiotherapy is commonly used to treat tumours within the body or skin of a human or animal patient. In such treatments, the cells forming part of the tumour are irradiated by ionising radiation in order to destroy or damage them. However, in order to apply a prescribed dose of ionising radiation to a target location or target region, the ionising radiation will typically also pass through healthy tissue of the human or animal body. It is desirable to minimise the dose received by healthy tissue in radiotherapy treatment.

The ionising radiation for radiotherapy is generated by a radiotherapy device such as a linear accelerator (LINAC). Such radiotherapy devices typically comprise rotatable components, such as a rotatable gantry with a radiation source attached thereto, to enable the radiation source to be rotated around a patient and thus for the radiation to be directed at the target area from a plurality of different angles. This ensures that the extent to which any given portion of healthy tissue, surrounding the target area, is exposed to potentially harmful radiation is minimised.

Radiotherapy devices such as LINACs typically also comprise a component such as a collimator, for shaping and directing radiation towards the target area, and away from healthy tissue. For example, a LINAC may comprise a multileaf collimator (MLC), which comprises a plurality of moveable ‘leaves’, which can be manipulated in order to shape a radiation beam in accordance with a particular patient's clinical need.

It is known to attach additional devices, referred to herein as ‘accessories’, to radiotherapy devices in order to provide finer control over the shape and/or directionality of the radiation beam. Examples of accessories that are commonly attached to radiotherapy devices include electron applicators, cones, and shadow trays.

An electron applicator is an accessory that can be used for electron beam radiotherapy. An electron applicator typically comprises a plurality of vertically-spaced substantially planar layers of a radiation-absorbing material, wherein each layer has an aperture through which the radiation beam can pass. In use, an electron applicator is positioned along the path of the radiation beam, between the radiation source and the patient, such that the beam passes through the aperture of each layer of the electron applicator, before reaching the patient. Electron applicators can provide additional collimation to the beam, and can also reduce the patient's exposure to scattered electrons and secondary radiation. Electron applicators having apertures of different sizes and shapes are available, such that, for operation of a radiation device to treat a patient, an electron applicator having a size and shape that is most suited to that patient's needs can be selected and attached to the radiotherapy device.

A cone is a block of a radiation-absorbing material comprising a bore through which the radiation beam can pass. In use, a cone is positioned within the radiation beam, such that the beam passes through the bore before reaching the patient. The bore further collimates the beam, such that the diameter of the beam when it emerges from the cone corresponds to the diameter of the bore. Cones having bores of different diameters are available, such that the cone that is best suited to a particular patient is selected and attached to the radiotherapy device.

A shadow tray is a device comprising a tray that can be positioned between the radiotherapy device and a patient. One or more blocks of a radiation-absorbing material, such as lead, are placed on the tray in order to prevent certain portions of the radiation beam from reaching the patient. The radiation beam is thus shaped according to the position of the blocks on the shadow tray. Shadow trays are typically employed when the radiation comprises an x-ray beam, as opposed to an electron beam.

Many radiotherapy devices, such as LINACs, for example the Elekta™ Digital Linear Accelerator, are configured to recognise the presence of an accessory attached thereto and to identify the accessory. For example, an electrical connection may be provided between the LINAC and the accessory, via one or more connectors having a plurality of pins, wherein at least some of those pins are configured to communicate a code or other identifier, from the accessory to the LINAC. In some cases, there may instead or also be a wireless communication link for identifying the accessory to the LINAC, or the accessory may be configured to identify itself to the LINAC another way. The LINAC may also be configured to detect or recognise when an accessory physically attaches thereto, via engagement components such as hooks, latches, connectors and so on.

A LINAC may also be configured to amend one or more of its operating parameters—for example, the size or shape of a diaphragm, and/or the configuration of leaves within an MLC—according to the type of accessory that is attached thereto. Amending the diaphragm or the leaves according to the identity (and therefore the characteristics) of the selected accessory can, for example, help to ensure that the aperture through which the radiation beam passes is of the correct configuration to ensure the required radiation field, for application of the radiotherapy to a target area. In order for an accessory to be employed successfully, the LINAC should respond to the presence of the accessory in an accurate, predictable and reliable manner.

In practice, a clinician can select an accessory for a radiotherapy session, in order to suit a particular patient's clinical need. Care must be taken to attach the correct accessory to the radiotherapy device for each radiotherapy session, because an incorrect accessory could result in the patient being exposed to an inappropriately-shaped beam or an inappropriate radiation dose.

It is desirable to ensure that a radiotherapy device operates accurately, to achieve patient safety and effective treatment of target areas, whilst protecting healthy tissue from the potentially damaging effects of radiation thereon. One aspect of this is ensuring that the LINAC (or other radiotherapy device) responds as expected, to the presence of a given accessory. It will be appreciated that, if the radiotherapy device were to respond in an unpredictable or inaccurate manner to the presence of a particular accessory, the resulting size and shape of the radiation beam applied to the target area may be incorrect. This could have significant and damaging effects for the patient, and would generally be regarded as being unacceptable from a clinical perspective.

Typically, manufacturers of radiotherapy devices and clinical radiotherapy providers will implement regulations—which in many jurisdictions arise from legal requirements—regarding the testing of the interactions between a LINAC (or other radiotherapy device) and the available accessories. Such regulations tend to be quite stringent, in order to ensure patient safety at all times. They may require regular ‘routine’ checks and additional checks when, for example, a hardware or software update is made to the LINAC and/or when the LINAC is repaired, installed, moved or relocated.

For example, a general standard that may apply is: ‘IEC 60601-1 Medical electrical equipment—Part 1: General requirements for basic safety and essential performance’. For example, a particular standard that may apply is: ‘IEC 60601-2-1 Medical electrical equipment—Part 2-1: Particular requirements for the basic safety and essential performance of electron accelerators in the range 1 MeV to 50 MeV’.

Testing the interactions between a LINAC (or other radiotherapy device) and the available accessories can be highly laborious and time consuming, as the number of different accessories that can be fitted to a single LINAC can be very large, and each accessory typically must be tested for multiple different beam energies. Moreover, individual accessories tend to be quite large and heavy, such that carrying out the testing is slow and physically demanding on the user. This can create restrictions on who can carry out the testing and on when it can be done, because at least one person needs to be present, throughout the testing process. These factors also restrict the availability of a LINAC for clinical use, as it cannot be used for providing radiotherapy treatment to patients at times when testing of the interaction between the LINAC and its accessories is being conducted.

### SUMMARY

An improved accessory for a radiotherapy device is provided, which enables the user to perform tests on a radiotherapy system in an efficient and streamlined manner, without compromising on system safety or accuracy.

The improved accessory (which may instead be referred to, for example, as an ‘accessory’ or a ‘test accessory’ or an ‘attachment’) may be configured to ‘emulate’ (or imitate, or mimic, or represent) a ‘real’ functional accessory (i.e. an accessory that can be used with a radiotherapy device, for providing radiotherapy treatment to a patient). For example, the improved accessory may emulate the electrical aspects of a particular real accessory. Therefore a radiotherapy device may perceive that a ‘real’ functional accessory is attached thereto, when the improved accessory is in fact attached. Tests can then be run to, for example, check and/or verify the responses of the radiotherapy device when it perceives that an accessory of a particular type or configuration has been attached thereto.

The improved accessory need not have all the beam attenuation hardware that a emulated ‘real’ accessory has, because the improved accessory is not intended to be used for clinical treatment of a patient This enables the improved accessory to be more compact and streamlined than a typical real accessory is. Therefore, physically handling the improved accessory may be less cumbersome and less time consuming than handling the ‘real’ accessory can be. Moreover, the improved accessory may be configured, or controlled, to emulate different respective ‘real’ accessories, at different respective times. Such control may be done remotely, and may be automated or semi-automated. This enables tests to be run, in which multiple different real accessories are emulated and the radiotherapy system's responses thereto checked and verified, in a time-efficient manner, requiring little or no user input during the tests.

According to an aspect, an accessory for a radiotherapy device is provided, comprising a first electrical connector for forming an electrical connection to a radiotherapy device, and a signal emitter configured to emit a signal in order for the accessory to emulate the connection of a second accessory, comprising a beam control apparatus, to the radiotherapy device.

The accessory may also comprise a first mechanical connector for forming a mechanical connection to a radiotherapy device.

The accessory may be a single piece item or may comprise two or more separate or separable components.

The signal emitter may be configured to emulate the connection of a second accessory to the radiotherapy device by sending, or by controlling another component or entity to send, a signal to the radiotherapy device. The signal may comprise data regarding one or more properties of the second accessory, the connection of which is being emulated. The signal may comprise the identity of the second accessory. The identity of the second accessory may be stored, in a list or library or database of accessory identities, by the radiotherapy device or by a memory that is accessible by the radiotherapy device.

The signal may be digital or analogue. The emulation of the connection, to the radiotherapy device, of the second apparatus may be automated or semi-automated. The emulation may be controlled by a computer or processor or by another suitable combination of hardware and software.

The signal emitter may comprise hardware and/or software. The signal emitter may be comprised within the first electrical connector. For example, the signal emitter may comprise one or more pins, within the first electrical connector. The signal emitter may be comprised within a controller that communicates with the first electrical connector. The signal emitter may be physically remote from the first electrical connector.

The accessory may be provided without one or more beam control components that the second accessory, the connection of which the accessory is configured to emulate, would comprise in practice, in order for it to be operable to provide beam control in conjunction with the radiotherapy device. The accessory may be configured to prompt the radiotherapy device, when it is in connection with the accessory, to carry out an action or provide a response as if the second accessory, including the one or more beam control components, were present. The connection between the accessory and the radiotherapy device may be any of mechanical and/or electrical and/or communicative.

The accessory may be further configured to update, or change, or vary at least one property of the second accessory, the connection of which the accessory is configured to emulate.

The first beam control apparatus, within the second accessory, may comprise any of an aperture, a collimator, a beam deflector, a beam absorber, or a block. The second accessory may be an electron applicator or a shadow tray.

For example, the second accessory may comprise an electron applicator, having an aperture, for example an end frame aperture. The accessory may be configured to emulate the presence of an end frame aperture of a particular identity, having a specific size and shape. The accessory may be further configured vary a property of the second accessory that it is emulating. For example, in this case it may be configured to vary, or change, or update, the size or the shape of the emulated end frame aperture.

For example, the second accessory, may comprise shadow tray, which comprises a block or a plurality of blocks, for example lead blocks. For example, the controller may be configured to emulate a shadow tray having a particular identity or number, which corresponds to a unique radiation absorbing, or arrangement of radiation absorbing, blocks. The first accessory may be configured to vary an emulated property such as the number or the arrangement or the size of the block or blocks.

The first mechanical connector may be configured for remotely-controlled actuation, in order to mechanically connect or disconnect the accessory to or from a radiotherapy device. The first electrical connector may be configured for remote control, in order to electrically connect or disconnect the accessory to or from a radiotherapy device. The mechanical and/or the electrical connection and/or disconnection may be controlled by a computer or a processor or by another suitable combination of hardware and software. The remote control of the mechanical connection and/or disconnection may be executed via actuation of one or more drive mechanisms or motors. The remote control of the electrical connection and/or disconnection may be executed via switching a switch and/or via the activation or deactivation of one or more electrical contacts.

A radiotherapy system may be provided comprising a radiotherapy device and an accessory according to the above aspect.

The radiotherapy system may further comprise a second accessory, wherein the second accessory comprises a second electrical connector for forming an electrical connection to the radiotherapy device, and a beam control apparatus. It may also comprise a second mechanical connector for forming a mechanical connection to the radiotherapy device,

The second beam control apparatus may comprise one or more beam control components that enable the second accessory to be operable to provide beam control in conjunction with the radiotherapy device.

For example, the second apparatus may comprise an electron applicator and the first accessory may be configured to emulate the connection of an electron applicator, to the radiotherapy device. For example, the second apparatus may comprise a shadow tray and the first accessory may be configured to emulate the connection of a shadow tray, to the radiotherapy device

The first mechanical connector, comprised within the first accessory, may be of the same type as the second mechanical connector, comprised within the second accessory.

The first electrical connector, comprised within the first accessory, may be of the same type as the second electrical connector, comprised within the second accessory.

The first accessory may share one or more mechanical and/or electrical properties with the second accessory. For example, the first accessory may have an outer size or shape that matches the outer size or shape of the second accessory. For example, the first accessory may attach to or detach from the radiotherapy device in a similar manner to the manner in which the second accessory attaches to or detaches from the radiotherapy device.

According to an aspect, a method is provided of testing a radiotherapy system comprising a radiotherapy device and a first accessory, wherein the first accessory comprises a first electrical connector for forming an electrical connection to the radiotherapy device and a signal emitter configured to emit a signal in order for the first accessory to emulate the connection of a second accessory, comprising a beam control apparatus, to the radiotherapy device. The method comprises connecting the first electrical connector of the first accessory, to the radiotherapy device and controlling the signal emitter to emit a signal in order to emulate the connection of a second accessory, comprising a beam control apparatus, to the radiotherapy device. The method further comprises testing a first operation of the radiotherapy device, with the first electrical connector connected thereto and with the emulated connection of the second accessory to the radiotherapy device.

The step of testing a first operation of the radiotherapy device may include observing, checking, verifying, measuring or recording a property or a behaviour of the radiotherapy device, during or as a result of the connection of the first electrical connector thereto and in the simulated presence of the second accessory.

The method may comprise, after testing the first operation of the radiotherapy device, updating at least one property of the second accessory, the connection of which is being emulated, and subsequently testing a second operation of the radiotherapy device, with the first electrical connector connected thereto and in the simulated presence of the updated second accessory. The updated second accessory may have a different identity to the initial second accessory. The updated second accessory may instead be referred to as a third accessory.

The method may further comprise, after testing the first operation of the radiotherapy device, and before testing a second operation of the radiotherapy device: disconnecting at least one of the first mechanical connector and the first electrical connector, from the radiotherapy device; updating at least one property of the second accessory, the connection of which is being emulated; and reconnecting the at least one of the first mechanical connector and the first electrical connector, to the radiotherapy device.

The disconnection and/or the reconnection of the at least one of the first mechanical connector and the first electrical connector, from or to the radiotherapy device may be remotely controlled.

The method according to the above aspect(s), or a method of controlling an accessory according to the above aspect(s), or a method of controlling a radiotherapy system according to the above aspect(s), may be a computer-implemented method.

According to an aspect, a computer program is provided comprising instructions which, when executed by data processing apparatus, causes the apparatus to perform a method according to the above aspect(s), or a method of controlling an accessory according to the above aspect(s), or a method of controlling a radiotherapy system according to the above aspect(s).

According to an aspect, a computer readable medium is provided storing a computer program according to the above aspect.

According to an aspect, a method is provided of designing a first accessory for use with a radiotherapy device, wherein the first accessory is intended for emulating the presence, in connection with the radiotherapy device, of a second accessory. The second accessory, the presence of which is to be simulated, comprises a beam control apparatus that that enables the second accessory to be operable to provide beam control in conjunction with the radiotherapy device. The method comprises identifying an intended purpose of the first accessory, in conjunction with the radiotherapy device. The method further comprises determining whether a component of the second accessory should be represented in the first accessory, in order to achieve the intended purpose of the first accessory, in conjunction with the radiotherapy device.

The step of determining whether a component of the second accessory should be represented in the first accessory, in order to achieve the intended purpose of the first accessory, in conjunction with the radiotherapy device, may comprise any of:
determining whether the component of the second accessory should be replicated within the first accessory; and/or
determining whether the component of the second accessory should be emulated within the first accessory; and/or
determining whether the representation of the component of the second accessory, within the first accessory, should be physically and/or functionally different to the actual component that is present within the second accessory; and/or
determining whether the component of the second accessory should be omitted from the first accessory.

The intended purpose of the first accessory may be, for example, to use it with a radiotherapy device for the purpose of conducting one or more tests.

The second accessory may also comprise a second mechanical connector for forming a mechanical connection to the radiotherapy device and/or a second electrical connector for forming an electrical connection to the radiotherapy device.

A first accessory may be provided, that has been designed in accordance with the above aspect.

It should be appreciated that the features of the above aspects are given by way of example. Except when they are explicitly mutually exclusive, any features described above in relation to any one of the aspects may be provided in any one of the respective other aspects.

### FIGURES

Specific arrangements are described herein, by way of example only, with reference to the figures, of which:

FIG. 1 shows a known type of electron applicator.

FIG. 2 shows the underside of the base plate of the known electron applicator of FIG. 1.

FIG. 3 shows a first face of an arrangement of a test accessory for a radiotherapy device.

FIG. 4 shows a second face of the arrangement of the test accessory of FIG. 3.

FIG. 5 shows a schematic representation of a controller for the test accessory of FIG. 3.

### DETAILED DESCRIPTION

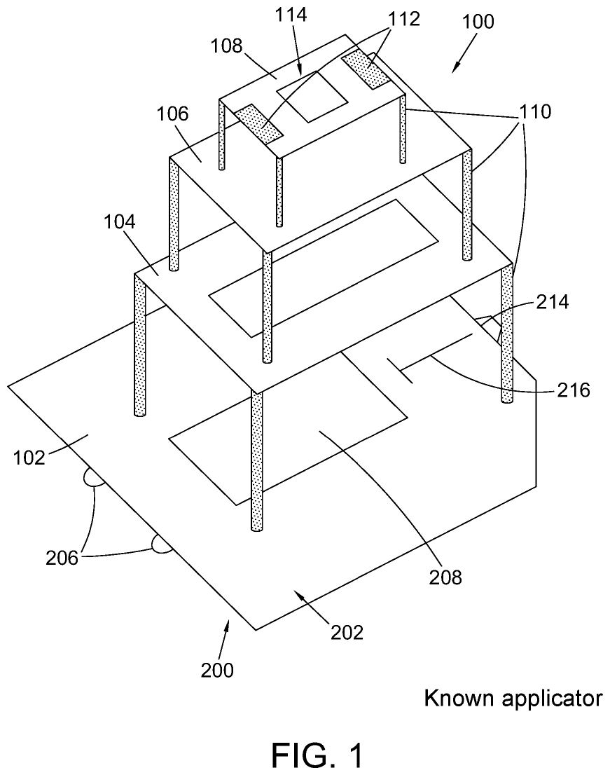

FIG. 1 shows an example of a known type of electron applicator 100. This electron applicator is configured for attachment to a LINAC, but it will be appreciated that attachment of electron applicators (and other accessories) to other types of radiotherapy devices is also possible.

The electron applicator 100 in FIG. 1 has a plurality of layers which comprise a base plate 102 and sequential first 104, second 106 and third 108 substantially planar stages. Each layer is physically connected to, but vertically spaced apart from, the respective next layer. The stages 104, 106, 108 and the baseplate 102 each comprise an opening or aperture substantially at its centre, configured for a radiation beam to pass through, when the electron applicator is used in attachment to a LINAC, for radiotherapy treatment. The outer cross-sectional size of the layers decreases, layer by layer, from the base plate 102 towards the third stage 108.

Although FIG. 1 shows the third stage 108 at the ‘top’ of the of the electron applicator 100, and the base plate 102 at the ‘bottom’, in practice it is the base plate 102 that mechanically and electrically connects the electron applicator 100 to a radiotherapy device. The third stage 108 is therefore the part of the electron applicator 100 that, in practice, will be located furthest from the radiation source on the LINAC and thus will be located closest to the patient. As the skilled reader will appreciate, the electron applicator 100 can attach at any suitable location, which enables it to guide and shape the radiation beam that exits the LINAC. For example, the base plate 102 of the electron applicator 100 may attach to a Beam Shaping Device (BSD) that forms part of the LINAC. The BSD may otherwise be referred to as the X-ray head, the MLC, or the Agility head.

The third stage 108 of the electron applicator may be configured to receive an end frame 114. For example, in the example shown in FIG. 1 there is a pair of slots 112 shown on a ‘top’ surface of the third stage 108 (the surface that will be closest to the patient, in operation) that can receive an end frame 114, which comprises removeable plate. This plate is usually in contact with the patient's skin during treatment. The aperture in the end frame 114 defines the area (on the patient) that will receive radiation. The dimensions of the end frame aperture therefore are what identify the ‘type’ of the electron applicator 100. For example, the end frame aperture may be square, rectangular or circular (or bespoke, as detailed further below). For example, the aperture could be 6 cm×6 cm, or 6 cm×14 cm, or 2 cm in diameter, or may have any other suitable dimensions and shape.

In known electron applicators, the default end frame shape is usually either square or rectangular. Each default end frame has a single hole drilled in its' underside. This single hole can be “read” by the applicator as a code of ‘1’. In reality, tumours are rarely square or rectangular, so it can be desirable to make a bespoke end frame for a particular patient, that has some other shape which matches the area to be treated. In order to ensure that the correct end frame is used during treatment of a particular patient, a combination of holes can be drilled or moulded on the underside of the bespoke end frame, to give a unique binary code. Any combination of 4 holes may be present allowing codes 0-15 inclusive. The patient's treatment prescription will specify the end frame needed for that treatment.

The electron applicator 100 has 4 microswitches (not shown in FIG. 1), each of which reads the presence, or absence of a coding hole using a spring-loaded ball. The combination of the 4 switches yields a 4-bit binary code, corresponding to the holes in the end frame. That is; Hole=1. No hole=0. Therefore, a real electron applicator can ‘read’ which end frame is received therein.

The connectors between sequential stages, and between the first stage 104 and the base plate 102, in this example comprise substantially vertical ‘legs’ 110, wherein each stage 104, 106, 108 is substantially square or rectangular in cross-sectional shape and has four legs 110 protruding therefrom, one located close to each of its four corners, extending towards the respective next stage 106, 108 or baseplate 102.

Although not shown in FIG. 1, the connectors, for example the legs 110, provided between the second 106 and third 108 stages may be spring mounted or otherwise biased, in order to provide scope for some movement or mechanical ‘give’ at the third stage 108, where the applicator 100 comes in contact with a patient. For example, there may be a spring mechanism provided between the second 106 and third 108 stages. Should the applicator be pressed too hard against the patient, the third stage 108 will move along the axis of the legs 110. One or more microswitches may be configured to detect this movement, signalling the Linac that a so-called ‘Touchguard’ event has occurred. The Linac may be configured to respond to this by preventing all movements of the Linac structure to prevent injury to the patient.

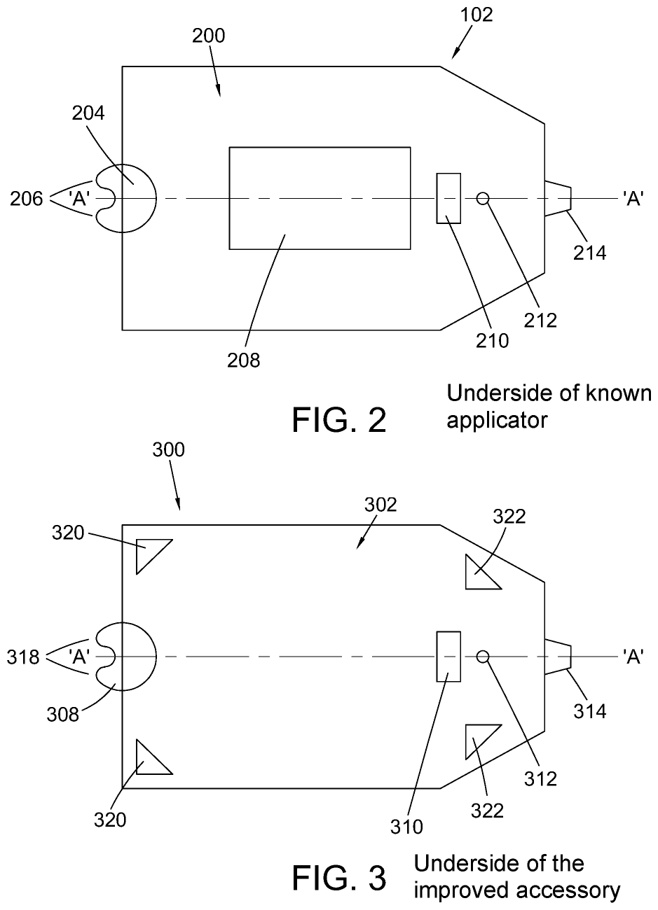

FIG. 2 shows the ‘underside’ 200 of the base plate 102 of the example electron applicator 100 of FIG. 1. The term ‘underside’ 200 in this context means the elongate face of the base plate 102 that cannot be seen in FIG. 1—as opposed to the ‘topside’ 202 of the base plate 102, which is visible in FIG. 1. As described above, the base plate 102 is configured to attach to a LINAC. The connectors for achieving such attachment are provided on the underside 200 of the base plate 102, in this example.

The underside 200 of the base plate 102 in this example comprises a hook 204 at one end of its elongate axis, ‘A’. The hook 204 comprises two prongs 206 that project substantially parallel to axis A, away from the main body of the base plate 102. The prongs 206 of the hook 204 are configured to be received by two slots or other receivers, on a compatible LINAC (not shown).

The baseplate 102 has a baseplate aperture 208, which enables radiation that exits the LINAC to travel through the applicator, towards the end frame 114 to the patient.

Further along axis A, the underside 200 of the base plate 102 comprises a first electrical connector 210. For example, the first electrical connector 210 may comprise an arrangement of pins, for example 24 pins, projecting out of the surface of the underside 200 of the base plate 102, substantially perpendicular to that surface. The first electrical connector 210 is configured to connect to a compatible electrical connector (not shown), provided on a LINAC. The first electrical connector 210 is configured to transmit electrical signals to the LINAC. It may also receive signals from other electrical components within, or in connection with, the electron applicator 100. For example, signals may be received at the first electrical connector 210 from other parts of the electron applicator 100, such as from the end frame 114, and it may transmit those signals, or some or all of the data comprised within those signals, to the LINAC. For example, it may transmit the signals identifying the end frame aperture type, and/or it may transmit signals regarding an event such as a ‘Touchguard’ event, as mentioned above.

In the example shown in FIG. 2, there is a pin 212 provided close to the first electrical connector 210, wherein the pin 212 projects out of the surface of the underside 200 of the base plate 102, substantially perpendicular to that surface. The pin 212 is configured to be received by a compatible hole or other receiver (not shown) on a LINAC. Its purpose is to help align the base plate 102 in the correct orientation, when it is being attached to the LINAC, as detailed below. The pin may be omitted or be replaced by an alternative alignment component or components, in other types of electron applicator or in other accessories.

At the distal end of the base plate 102, substantially opposite the hook along the elongate axis, A, there is a retractable latch 214. The retractable latch 214 can be configured to project substantially parallel to axis A, away from the main body of the base plate 102 (in the opposite direction to the direction in which the prongs 206 of the hook 204 project). The retractable latch 214 is configured to be received by a compatible receiver (not shown) on a LINAC, to form a mechanical attachment between the distal end of the base plate 102 and the LINAC.

In operation, the example electron applicator 100 of the type shown in FIGS. 1 and 2 can be manually fitted to a LINAC via the following three step sequential process:
1. The hook 204 at the first end of the base plate 102 is received by compatible receivers on the LINAC.
2. The first electrical connector 210 is connected to a compatible electrical connector on the LINAC.
3. The retractable latch 214 is aligned with its compatible receiver on the LINAC. The latch 214 can then be extended, to be received by the compatible receiver and lock the electron applicator in place.

As a result of the above three steps, the electron applicator 100 is mechanically and electrically connected to a compatible LINAC. The LINAC should, once fitting of the electron applicator 100 is complete, ‘realise’ that the it has been fitted—for example, the LINAC may comprise one or more microswitches for this purpose. The electron applicator 100 will typically provide the Linac with the following information: Accessory Type (Shadow Tray, fixed Applicator, etc), Aperture Type (square, rectangular, circular, etc), X-size, Y-size, Applicator Present, Touchguard status, end frame code number. Other types of accessory (such as shadow trays) may provide other information to the LINAC.

Once it realises that the attachment has been made, and has received information from the electron applicator 100, the LINAC should amend any of its operating parameters accordingly. For example, the diaphragm of the LINAC may adjust its size or shape, to be compatible with the electron applicator 100. For example, it may be that the currently selected beam is inhibited if the fitted accessory is incompatible (e.g. if X-rays are currently selected, then an electron applicator is not compatible with this modality). If the user selects an electron energy, then the Linac will automatically set up the correct field size, according to previously learnt and stored settings on the Linac.

The Linac can subsequently be used for radiotherapy treatment (or for testing), working in conjunction with the election applicator.

In this example, because the pin 212 is located close to the first electrical connector 210, the pin 212 will be received by a compatible hole on the LINAC, to assist with aligning the distal end of the base plate 102, at substantially the same time as the first electrical connector 210 is connected to its compatible electrical connector. But in other known electron applicators there will be no pin, or the pin will be at a different location, or there will be an alternative component provided for aligning the applicator correctly on a radiotherapy device.

As mentioned above, in this example of a known electron applicator 100, the prongs 206 of the hook 204 are configured to be received by two slots or other receivers, on a compatible LINAC (not shown). The LINAC may comprise an overhanging member, such as a shelf, that extends over the two slots so that, when the prongs 206 are received in the slots, the shelf prevents them from disengaging from the slots. In this example, the baseplate 102 may therefore have to be directed towards the LINAC at an angle, in a substantially sideways and downwards action, in order for the hook 204 to clear the overhanging member and be received in the slots.

Once the hook 204 has been received, in this example, the rest of the base plate 102 can be lowered down towards the LINAC, in a substantially pivoting movement, whereby the first electrical connector 210 will come into the contact with the LINAC first, then the pin 212, then the latch 214. Once the latch 214 is in contact with the LINAC, the entirety of the base plate 102 should be in place, and so it can be locked or otherwise fixed in position. In this example, there is a handle 216 provided on the topside 202 of the base plate 102 (which is visible in FIG. 1), which can be used to extend the latch 214 and thus to lock the base plate 102—and therefore to lock the electron applicator 100—into place, on the LINAC.

When the electron applicator 100 is to be released from the LINAC, a disengagement process should be followed, which is the reverse of the above-described fitting process. That is; the disengagement for this example should follow these three sequential steps:
1. The latch 214 is retracted, to be released by the compatible receiver and unlock the electron applicator 100. The user can then begin to lift the electron applicator 100 away from the LINAC.
2. The first electrical connector 210 is disconnected from its compatible electrical connector on the LINAC.
3. The hook 204 at the first end of the base plate 102 is released from its compatible receivers on the LINAC.

Again, for this example, the electron applicator may be pivoted about the hook 204 and then lifted in a sideways and upwards motion, away from the slots that receive the hook 204, in order to clear the overhanging member that keeps the hook 204 in place, on the LINAC (not shown). Also in this example, the pin 212 will be disengaged at around the same time as when the first electrical connector 210 is disengaged.

It will be appreciated that the above description is of one known example of an existing electron applicator. Other electron applicator types also exist, that differ from the above described example. For example, some electron applicators have fewer or more layers than the above-described example. For example, the layers may be of a different shape or size than those described above and depicted in FIGS. 1 and 2 herein. For example, the connectors between layers may look different to those that are shown in, and described in relation to, FIGS. 1 and 2 herein.

The mechanical and electrical components via which some known electron applicators and/or other accessory types engage with radiotherapy devices may differ from those that are shown in, and described in relation to, FIGS. 1 and 2 herein. For example, they may not have a hook and/or a latch and/or a pin. For example, they may have mechanical engagement components that are integral to the base plate and/or that are attached to the base plate via a screw fit or via any other suitable mechanism. For example, they may have more than one electrical connector and/or they may have different types of electrical connectors and/or they may communicate signals to the LINAC via another component such as via a USB cable, or via a wireless protocol or via any other suitable connector. For example, they may be single piece items or may comprise a plurality or individual pieces or component parts.

All the above notwithstanding; regardless of the particular mechanical and/or electrical and/or signalling details of existing electron applicators—and, indeed, of other accessories for use with radiotherapy devices, such as shadow trays—a common drawback of existing radiotherapy systems that employ accessories in conjunction with a radiotherapy device is that the accessories tend to be many in number and are generally relatively large and cumbersome to transport and can be time consuming to fit to, and release from, the radiotherapy device. This can be exacerbated by the fact that a person should not be in the same room as a radiotherapy device, when it is emitting a radiation beam, unless that person is a patient for whom the beam is intended, due to the potentially dangerous effects of the radiation on healthy human tissue.

An improved accessory is described herein. The improved accessory may be used to overcome the above, and other, drawbacks of existing radiotherapy systems, particularly with respect to testing operation of a radiotherapy system when it is not in use for treating a patient. The improved accessory may also be provided for use in conjunction with future developments of radiotherapy systems, and their respective accessories. The improved accessory will be referred to herebelow, for ease of reference, as a ‘test accessory’, however this term should not be regarded as being limiting.

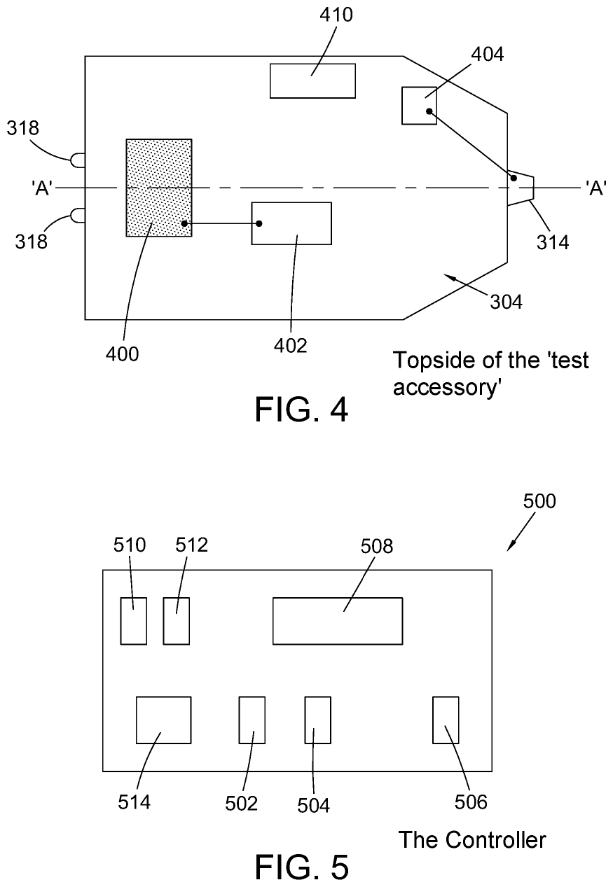

The test accessory can be configured to emulate specific ‘real’ functional accessories, which are compatible for use with a particular LINAC or other radiotherapy device, for testing purposes. For example, as will be described in detail herebelow in relation to FIGS. 3 and 4, the test accessory can be used to emulate accessories of the type shown in FIGS. 1 and 2 herein, for testing purposes. But this is just one example of how the test accessory can be physically implemented. Just as the accessories that are used for radiotherapy treatment can vary in function and form; the test accessory can also be created in different ways, in order to successfully emulate those accessories for testing purposes.

For example, the test accessory can be used to emulate ‘real’ functional applicators and/or ‘real’ functional shadow trays, for test purposes.

A single test accessory may be provided in order to emulate multiple different ‘real’ functional accessories, such that multiple different respective tests may be performed without the need to change the physical entity that is attached to the radiotherapy device, between each test. Furthermore, aspects of the test accessory that need to change in order for it to represent different respective accessory types may be changed remotely and may in some cases be changed automatically, for example under pre-programmed computer control. This opens up the possibility of conducting tests for a radiotherapy device in the absence of a user (at least for the majority of the testing time) and, for example, opens up the possibility of conducting tests at times that up until now have been very difficult or user-unfriendly, such as overnight. As a result, the radiotherapy device can be more available at times at which it is likely to be in demand for use in treating patients, thus making it more useful to the clinician and more efficient, overall.

It has been recognised herein that, in order to test a radiotherapy device such as (but not limited to) a LINAC and, in particular, to test the manner in which it interacts with its accessories, it is not necessary to provide all the physical and functional components of every accessory, in order to achieve reliable and accurate test results. Instead, certain selected components (or aspects) of the accessory may be replicated, certain other selected components (or aspects) may be omitted and certain other selected components (or aspects) may be emulated, in order to provide an imitation of an accessory, which can interact with a corresponding radiotherapy device. These recognitions and selections may be physically realised in the test accessory, detailed herein.

The components or aspects of a ‘real’ functional accessory that are needed for a test may be emulated by the test accessory in any suitable manner. The manner in which particular components or aspects of a functional accessory are emulated by a test accessory—and, indeed, the selection of which components are replicated, which are emulated, and which are omitted—may vary according to the specifics of that accessory or group of accessories, and/or according to the test(s) that is/are to be carried out, using the test accessory.

For example, it is typical to perform regular tests, to check and verify the manner in which a radiotherapy device responds to the attachment of one or more of its accessories thereto. Such tests may look at, for example; whether the radiotherapy device recognises that an accessory has been attached thereto; whether the radiotherapy device can identify the attached accessory; and/or whether the radiotherapy device can accurately adjust its operating parameters to be compatible with the identified accessory, in order to ensure correct subsequent operation of the device for radiotherapy treatment.

It has been recognised herein that if, for example, a test concerns how a LINAC responds to the attachment of an electron applicator thereto, the collimation of the beam by the electron applicator is not relevant—because the beam is not being used for treatment or imaging purposes during the test, and the collimation itself is not what is being tested. Therefore, it has been recognised herein that there is no need to provide a complete, functional electron applicator—with all its physical layers, which are for collimation purposes—for such a test.

Instead, what may be needed for test purposes can include, for example:
the connection of an electrical connector, via which the LINAC could receive signals indicating the (emulated) presence of a particular electron applicator; and
the transmission, to the LINAC, of one or more signals identifying the (emulated) electron applicator to the LINAC.

The physical attachment of an engagement component that would activate a corresponding microswitch (or other component) of a LINAC, to indicate that an accessory is attached, may also be needed, in some arrangements.

In order to emulate one or more ‘real’ functional accessories, a test accessory may comprise physical components that are the same as (or similar to) corresponding physical components on a functional accessory. This may enable the test accessory to physically and/or electrically engage with a radiotherapy device in the same (or in a similar) manner as the functional accessory does, without requiring any changes to be made to the radiotherapy device. This may be particularly suitable if, for example, those physical components are common to a plurality of different functional accessories.

For example, if a plurality of functional electron applicators all have the same (outer) size and shape of base plate, and/or all feature the same physical engagement or locking mechanisms for attachment to a LINAC, it may be determined that the test accessory should also feature the same size and shape of base plate and/or should also feature the same physical engagement or locking mechanisms for attachment to a LINAC. However, it may be determined that the manner in which the physical engagement or locking mechanisms are actuated should be different on the test accessory, as compared to on the functional electron applicators. For example, it is typical for known functional electron applicators to be manually fitted to a LINAC, as described hereabove in relation to FIGS. 1 and 2. It may be determined that the physical engagement or locking mechanisms on the test accessory should instead be fitted automatically, for example under computer control, to better facilitate autonomous testing, in the absence of a user. This may be further understood from the detailed example given below, in relation to FIGS. 3 and 4 herein.

For example, if a functional electron applicator is a single-piece item, such as in the example of FIGS. 1 and 2 herein, the test accessory that emulates it may also be a single-piece item. Alternatively, the test accessory may comprise multiple separate parts. For example, there may be a mechanical connector that is physically separate to the electrical connector of the test accessory. In some arrangements, it may be possible to omit a mechanical connector from the test accessory and instead to configure the LINAC either not to require a mechanical connection, if it knows it is working in a ‘test’ mode, or it may be possible for the electrical signals conveyed by the test accessory to activate a switch, within the LINAC, that would ordinarily be activated by the mechanical attachment of a ‘real’ functional accessory.

A test accessory may also comprise components that emulate the presence of corresponding components of a functional accessory. For example, it may be determined that emulation is appropriate for certain components if the responses, of the radiotherapy device, to the presence of those components should be checked or verified, but the actual physical presence or operation of those components is not actually required, for testing purposes.

For example, when a functional electron applicator is attached to a LINAC, the LINAC recognises or identifies its end frame aperture size. The LINAC may make this recognition/identification based on a signal that it receives from the electron applicator, or from another source. The LINAC may then adjust its own operating parameters (such as diaphragm size/shape and modality) in order to ensure that it works correctly in conjunction with that electron applicator. A LINAC should typically be pre-programmed so that, when it receives a signal identifying the presence of a particular type of accessory attached thereto, it can automatically adjust its operating parameters, bearing in mind factors such as the energy of the beam that is to be applied and the particular needs of the patient and the target area to be treated. In some cases, there may be some user control involved in the adjustment of those operating parameters.

It has been recognised herein that, since the radiation beam is not actually being used for patient treatment during testing, the provision of end results such as a flat radiation field is not required during testing per se. Instead, what matters for the test purposes in this example is whether the LINAC can adjust its parameters correctly, in response to a received signal indicating that the end frame aperture is of a particular size and shape. Therefore, it is not necessary for the test accessory to actually have an end frame with an aperture of a particular size or shape. Instead, it is sufficient for an identifying signal to be conveyed to the LINAC, to indicate that the test accessory has an end frame aperture of a predefined type. The signal conveyed to the LINAC, by the test accessory, should be of a type that matches the signal that a ‘real’ functional electron applicator of that type would send to the LINAC, during normal operation. As mentioned above, the LINAC should typically be programmed to recognise and respond to such a signal by adjusting its operating parameters accordingly. With the test accessory in place, the signal can be conveyed to the LINAC and the response of the LINAC to that signal—for example, its adjustment of operating parameters—can be checked, verified, measured, recorded, or otherwise tested.

If the test accessory is configured to emulate the presence of corresponding components of a functional accessory, it may be configurable to emulate different components at different respective times. For example, if the presence of an aperture of a given size or shape is emulated, by sending an identifying signal of an appropriate type to the LINAC, it may be possible to change that signal and therefore to test the LINAC's response to different respective applicator end frame aperture sizes and shapes, without replacing or swapping the physical entity that is attached to the LINAC, between tests. For example, the details of the identifying signal(s) emitted by (or on behalf of) the test accessory may be changed under computer control, which may be automated or may require some user input, as will be understood further from the example detailed below in relation to FIGS. 3 and 4 herein.

An arrangement of a test accessory is shown, by way of example, in FIGS. 3 and 4. It will be appreciated that FIGS. 3 and 4 show one example of how a test accessory of the type described herein can be physically realised. However the test accessory may take other physical forms, whilst still providing the advantages described herein. The test accessory in this example emulates an electron applicator but it, or another test accessory, may be used to emulate other accessory types, for example shadow trays.

The test accessory 300 shown in FIGS. 3 and 4 is configured to emulate, or replicate or mimic, the electron applicator 100 (or a group of such electron applicators 100) that is shown in FIGS. 1 and 2, for testing purposes. The test accessory 300 in this example is therefore configured for attachment to a LINAC (not shown), of the type that the electron applicator 100 in FIGS. 1 and 2 can attach to. However the test accessory 300 shown in FIGS. 3 and 4 is not intended for, and not generally suitable for, use in conjunction with a LINAC for the provision of radiotherapy treatment to a patient. This is because, for example, the test accessory 300 in this example does not include collimation components, such as the first 102, second 104 and third 106 stages that are provided in the electron applicator 100 of FIGS. 1 and 2.

FIG. 3 shows a view of the ‘underside’ 302 of the test accessory 300. The term ‘underside’ 302 in this context means the elongate face of the test accessory 300 that comes into contact with, and at least in part attaches to, the LINAC. It is therefore configured to emulate the ‘underside’ 200 of the base plate 102 of the electron applicator 100, which is visible in FIG. 2.

FIG. 4 shows a view of the ‘topside’ 304 of the test accessory 300. The term ‘topside’ 304 in this context means the elongate face of the test accessory 300 that will remain visible to the user when the test accessory 300 is attached to a LINAC (not shown). It is therefore configured to emulate the ‘topside’ 202 of the base plate 102 of the electron applicator 100, which is visible in FIG. 1.

As can be seen from FIGS. 3 and 4, the outer size and shape of the test accessory 300 is the same as the outer size and shape of the base plate 102 of the electron applicator 100 of FIGS. 1 and 2. Therefore, the test accessory 300 fits into the same receiving region of the LINAC as the electron applicator 100 does.

The test accessory 300 has a hook 308, a first electrical connector 310, a pin 312 and a latch 314. These features are sized, shaped and orientated in order to emulate the respective sizes, shapes and orientations of the hook 204, first electrical connector 210, pin 212 and latch 214 that are provided on the electron applicator 100, as described in detail in relation to FIGS. 1 and 2 herein. Therefore the test accessory 300 can physically attach to the LINAC in a manner that, from the LINAC's perspective at least, emulates the attachment of a ‘real’ functional electron applicator to the LINAC. However, in this example, the manner in which that attachment is controlled can be remotely controlled, for example using a computer, as opposed to being manually controlled. This can be understood further in relation to FIG. 4.

It can be seen that the test accessory does not have the equivalent of a base plate aperture. This is because it is not necessary for a radiation beam to travel through (or via) the test accessory. However in some arrangements the test accessory could have an aperture therein.

The test accessory 300 also has two sets of magnets, which in this example are Neodymium magnets 320, 322. The first set of magnets comprises a pair of ‘hook end magnets’ 320, which are arranged on the underside 302 of the test accessory 300, close to the corners at the same end as the hook 308. The second set of magnets comprises a pair of ‘latch end magnets’ 322, which are arranged on the underside 302 of the test accessory, close to the corners at the same end as the latch 314. These magnets 320, 322, are provided for keeping the test accessory 300 in place on the LINAC, during an automated detachment and reattachment process (described below), when the hook 308, latch 314 and pin 312 will be temporarily detached from the LINAC. In effect, therefore, the purpose of the magnets 320, 322 is to ensure that the test accessory does not fall off the LINAC, during an automated detachment.

Other means of keeping the test accessory attached to the LINAC during an automated detachment and reattachment process may instead be used, in other arrangements. For example, support cradles may be provided on the topside 304, which are for receiving a clamp or other mechanism, which can fix the test accessory to the LINAC. Other arrangements may have respectively other components for fixing the test accessory 300 in place on the LINAC, during an automated detachment and reattachment process. Or in some cases it may not be necessary to provide an additional component for fixing the test accessory 300 in place on the LINAC, during an automated detachment and reattachment process.

As can be seen in FIG. 4, at one end of the topside 304 of the test accessory 300, close to the hook 308, there is a first drive mechanism 400 that is connected to a first motor 402, wherein the first motor 402 is configured to control the first drive mechanism 400 to actuate the hook 308. The first motor 402 can be driven under control of a computer or other processor, in order to achieve automatic (or at least partially automatic, in some cases) actuation of the hook 308. There is a second motor 404 provided, for driving a second drive mechanism that is connected to the latch 314, at the opposite end of the test accessory 300. Again, the second motor 404 can be driven under control of a computer or other processor, in order to achieve automatic (or at least partially automatic, in some cases) actuation of the latch 314.

There is a second electrical connector 410 provided on the topside 304 of the test accessory 300. In some arrangements, there may be multiple electrical connectors provided and/or at least one of the connectors provided may take a different form, other than that which is shown in FIG. 4. A role of the second electrical connector 410 is to provide an electrical (and communications) link between the first electrical connector 310—which connects to the LINAC—and a controller (not shown in FIG. 4), which is discussed further below. The second electrical connector 410 also provides an electrical (and communications) link between other aspects such as the first 402 and second 404 motors and a controller (not shown in FIG. 4).

Although FIGS. 3 and 4 show features such as the hook being exposed and visible, in practice one or more covers or casings may be provided, to protect such features.

FIG. 5 shows a schematic representation of a controller 500 that may be used for controlling the test accessory 300. It will be appreciated that this is just one example, and that other controllers may instead be used for controlling a test accessory—either of the type shown in FIGS. 3 and 4, or of another type—in accordance with the principles described below.

In general terms; the controller 500 is configured to send signals to, and receive signals from, the test accessory 300, and to send signals to the LINAC via the test accessory 300, in order to enable the test accessory 300 to emulate, or mimic, one or more ‘real’ functional accessories, at different respective times, for test purposes.

For example, the controller 500 can emit signals indicating that the test accessory 300 comprises end frame apertures of multiple different sizes and shapes, at different respective times, for the running of multiple respective tests. Moreover, in this arrangement at least, the controller 500 is configured to control the motors 402, 404 on the test accessory 300 to actuate the mechanical (dis)engagement components on the test accessory 300, and to control the switching on and off of the first electrical connector 310, in order to enable the test accessory 300 to recreate the steps that a ‘real’ functional accessory goes through during manual attachment and detachment to and from the LINAC. Therefore the controller 500 can enable the test accessory 300 to effectively make the LINAC ‘think’ or perceive that different ‘real’ functional accessories are being attached and detached, between respective tests, when in fact the same test accessory 300 can remain in place throughout those tests, with any controls being provided via the controller 500, either automatically or with some user input at the controller 500.

The particular example of a controller 500 shown in FIG. 5 comprises a first relay board 502 and a second relay board 504. The possible functions and compositions of relay boards will be well known to the skilled reader. In this example, the first relay board 502 forms an ‘isolation layer’ in which all relays may be switched on and off simultaneously, in order to connect or disconnect all the pins of the first electrical connector 310 on the test accessory 300 to or from the LINAC, simultaneously (or substantially simultaneously). Hence, it enables the test controller 300 to emulate an electrical connector of a ‘real’ functional accessory being physically connected and disconnected from the LINAC.

The second relay board 504 is configured to provide signals representing different possible functions and identities of ‘real’ functional accessories. The second relay board 504 therefore enables the test accessory 300 to emulate any valid (or, in some cases, invalid) accessory such as an electron applicator or a shadow tray and so on. For example, one or more relays within the second relay board 504 may emulate one or more pins of a connector on a ‘real’ accessory. Whereas in the real accessory, the pins could, for example, be connected to OV to represent logic ‘0’, or 10V representing logic ‘1’, and thereby enable groups of pins to produce binary numbers to identify information to the LINAC; in the test accessory those binary numbers could instead be produced by switching relays. The skilled reader will know how to implement such an arrangement, in practice. The skilled reader will also appreciate that relay boards of this type are only one example of how electrical connections for a real accessory such as an electron applicator could be emulated, by a suitable controller.

The controller 500 in FIG. 5 comprises a USB interface 506, for connecting to a computer or other processor. But another interface may be provided instead of or as well as a USB interface.

The controller 500 in FIG. 5 comprises a 10V reference supply 508, which acts as a fold-back circuit, to protect the electronics in the event of a power surge. Again, the skilled reader will appreciate that it is generally wise to provide fault protection in any electronic set-up and that the particulars of the fault protection provided for a test accessory can be changed or configured as appropriate, on a case-by-case basis.

The controller 500 in FIG. 5 comprises first 510 and second 512 digital potentiometers. The first 510 and second 512 digital potentiometers are configured to output specific voltages in response to commands from a microcontroller 514 in order to represent, respectively, the ‘x’ and ‘y’ dimensions (i.e. the dimensions along first and second mutually perpendicular axes) of the end frame apertures of different electron applicator types. The digital potentiometers 510, 512 can be controlled to vary the dimensions, to enable the test accessory 300 to emulate multiple different ‘real’ functional electron applicators at different respective times. Hence, tests can be run so that the LINAC's responses to different electron applicators, at one or more different beam energies, can be checked and verified (for example, using a comparison to stored predefined data) using a single test accessory 300 and without the need for a user to manually move or adjust that test accessory 300 between or during such tests.

The controller 500 in FIG. 5 also comprises the microcontroller 514. Again, it will be appreciated that other types of controller or processor may be implemented within a control unit for a test accessory, in accordance with the principles described herein.

The controller 500 may be configured to be self-checking. For example, a feedback loop may be implemented wherein a user inputs a control command (via e.g. a GUI, as detailed further below) and the controller may be configured to carry out whatever action the user has commanded and also to check that, as a result of that action, the test accessory is indeed operating (or configured to operate) as per the user's command. That is; the controller 500 may be configured not only to accept and respond to user commands but to self-check that it is responding correctly.

The controller may have other features, not specifically shown in FIG. 5. For example, it may comprise relays or other components that can enable fault testing. For example, fault situations such as a broken wire or dirty or interrupted connection, may be simulated. For example, the test accessory may be able to emulate incorrect codes, such as incorrect end frame codes, to test a LINAC's reaction thereto. Incorrect voltages or changing voltages may also be output by, for example, the digital potentiometers, to test whether they are detected by the LINAC.

A controller may vary in its features, according to the type of real functional accessory that is to be emulated by the test accessory. For example, digital potentiometers may not be needed for a controller of a test accessory that only emulates shadow trays. A controller may comprise other features, such as microswitches or any other suitable components, in order to send appropriate controls to a test accessory, and receive signals therefrom, in order to control its operation for emulating real accessories, for test purposes.

As mentioned above, the controller 500 can connect, via wired and/or wireless connection, to a computer or other processor. For example, the processor may comprise, or be comprised within, any suitable machine or device including, but not limited to, a PC, laptop computer, tablet, Smartphone and so on. We will refer to the processor by the general term ‘computer’ in this description, for ease of reference, but this should not be regarded as being limiting.

In some cases, the ‘controller’ 500 will not be physically separate to the ‘computer’. A single controller or processor may instead be employed, which can receive user inputs and/or be programmed to execute automated control of the operation of a test accessory.

The ‘computer’ in this example has a graphical user interface (GUI) (not shown), via which a user can provide inputs for controlling use of the test accessory 300. It will be appreciated that other types of user input interface may be used instead of or as well as a GUI, for this purpose. In some arrangements, the test accessory 300 and its controller 500 can comprise a ‘headless’ design that allows different user interface software to utilise the hardware and firmware of the test accessory 300 and controller 500, to operate them. Moreover, the ‘headless’ design may allow for either manual and/or automated control of the operation of the test accessory 300 and controller 500.

The GUI may receive user inputs via, for example, a real or virtual keyboard, manipulation of a mouse, touchscreen inputs and so on. It may comprise regions via which a user can select or otherwise input a command, and regions that can display information regarding the test accessory 300. For example, there may be an ‘applicator’ input, via which the user can select an applicator type, from a list of possible applicator types and configurations (i.e. aperture sizes)

The GUI may also include icons or regions that enable the user to input commands to remotely remove or fit the test accessory 300 from or to the LINAC. Such commands prompt the controller 500 to control the motors 402, 404 to actuate the hook 308 and the latch 314 of the test accessory 300, to disengage or engage as appropriate, in a manner that substantially replicates the actions that a user would follow when manually removing or fitting an accessory from or to the LINAC. Such commands may also prompt the controller 500 to electrically connect or disconnect the first electrical connector 310 to or from the LINAC, by switching the first relay board 502 accordingly.

In this example, the controller 500 is configured to ensure that the hook 308, the first electrical connector 310 and the latch 314 are fitted/connected in sequence, in order to exactly replicate the sequence of events that takes place when a user manually fits a ‘real’ functional applicator to the LINAC, as described in relation to FIGS. 1 and 2 herein. But it will be appreciated that this may not always be required. In general terms, the controller should usually be configured to control mechanical and electrical (dis)engagement of a test accessory using whatever steps, and involving whichever component parts of the test accessory, that are needed to replicate the steps and component parts involved in fitting the ‘real’ accessory that is being emulated, to the corresponding radiotherapy device. In some arrangements, it may be possible to alert the radiotherapy device that it is in ‘test’ mode, which may remove the need to replicate a manual fitting process exactly—for example, the sequence in which various components are fitted or connected may not matter, or it may be sufficient for the test accessory to emulate just the electrical connection(s) that the real accessory makes, and not also emulate its mechanical connections.

The GUI may be configured to provide visual feedback to the user, to confirm the inputs that he or she has made and to confirm what actions have occurred as a result. For example, it may provide confirmation when a hook and/or an electrical connector and/or a latch have been mechanically engaged, in response to the user inputting a ‘fit command.

The GUI can be displayed on any suitable hardware and may be comprised within any suitable computer program or software package. For example, it may form part of software that is stored locally on the computer or other processor, or on a portable memory, or on a server or cloud memory. The GUI may be accessible via an application (app), or via a URL, or by any other suitable route.

Thus, it can be seen that a computer may be used to remotely control the operation of the test accessory 300, for use to emulate (or replicate) the use of multiple different ‘real’ functional accessories, one at a time, for testing or verifying the interactions between a radiotherapy device, such as a LINAC, and those multiple different ‘real’ functional accessories.

Typically, the steps involved in using the test accessory 300 for such tests are as follows:
a. Before the tests begin, manually put the test accessory 300 in place on the receiving area of the LINAC (i.e. on the area of the LINAC that usually receives ‘real’ functional accessories). For example, in the particular example shown herein, the pin 312 of the test accessory 300 can be received in the compatible receiver on the LINAC, to assist with alignment.
b. If required), apply an additional clamp or other fixing mechanism, to prevent the test accessory 300 from moving or falling away from its position on the receiving area.
c. Ensure that the test accessory 300 is connected to its controller 500, which in turn should be connected to the computer (i.e. to the processor that will control operation of the test accessory 300, during the tests), if the controller and computer are provided as two separate entities.
d. Use the computer to apply the appropriate settings for the first test—for example, selecting the aperture size that the test accessory 300 should emulate.
e. Once the settings have been applied, use the computer to attach the test accessory 300 to the LINAC. In the particular example described herein, this will cause the hook 308, the first electrical connector 310 and the latch 314 to be fitted/connected, in sequence, to emulate the order in which those three components would fit/connect to the LINAC if a user were placing a ‘real’ functional electron applicator (or, indeed, the test accessory 300) on the LINAC manually.
f. Run the first test, to assess how the LINAC responds to the fitting and connection of the test accessory 300 thereto.
g. When the first test is complete, use the computer to remove (or emulate removal of) the test accessory 300 from the LINAC. In the particular example described herein, this will cause the latch 314, the first electrical connector 310 and the hook 308, to be removed/disconnected, in sequence, to emulate the order in which those three components would be removed/disconnected from the LINAC if a user were manually removing a ‘real’ functional electron applicator (or, indeed, the test accessory 300) from the LINAC. In this example, the magnets 320, 322, clamp or other fixing mechanism will ensure that the test accessory 300 does not fall off the LINAC, but the LINAC will perceive that the applicator, that it previously had perceived as being attached thereto, has been removed.
h. Steps d. to g. can then be repeated, for a plurality of different settings, changed under computer control, to enable the test accessory 300 to emulate different respective ‘real’ functional accessories, one at a time, that may include electron applicators and/or shadow trays, and for the LINAC's responses thereto to be checked and verified.
i. When the testing is complete, the test accessory 300 can be manually removed from the LINAC.

It will be appreciated that a test of a LINAC's response to the computer-controlled fitting/connecting of the test accessory 300 (in its guise as a particular electron applicator) may be run during the fitting/connecting process and/or after it has been fitted and connected. The ‘test’ may be a plurality of tests. The test(s) may be run in any suitable manner, dependent on the details of the LINAC and of the responses that are to be checked and verified. For example, the test(s) may comprise checking the positions of the collimator leaves and/or the diaphragm(s), once it has responded to the presence of the test accessory (which the LINAC will have perceived as being an electronic applicator having the selected aperture size.) For example, the test(s) may comprise checking the configuration of a diaphragm as compared to one or more reference values or reference characteristics, which the LINAC diaphragm would be expected to exhibit for the particular applicator that is being emulated, for a given beam energy. For example, the response of the LINAC to the test accessory, in its guise as an emulated functional accessory, may be compared to a previously-recorded response of the LINAC to the actual presence of that functional accessory. The test(s) may be repeated for multiple different beam energies for each accessory, which is being emulated—however it may be sufficient to change a setting to indicate a change in beam energy, as opposed to actually running the beam. The test(s) may be fully automated or may require some user input. The test(s) may be run via the same computer that controls operation of the test accessory 300, or may be run via a separate processor.

Although a GUI has been described herein, via which a user may select and apply particular settings, in some arrangements the testing of the responses of a LINAC or other radiotherapy device to multiple different accessories, as emulated, one by one, by a test accessory such as the one described herein, at one or more beam energies, may be run at least partially automatically. That is; a ‘computer’ or other controller or processor may be configured to run through a sequence of tests, varying one or more settings of the test accessory between respective tests, in an autonomous fashion.

As described in detail hereabove, a test accessory can be configured to stay in place on the radiotherapy device, whilst emulating the steps of manual attachment and detachment that the radiotherapy device would recognise for its ‘real’ functional accessories. For example, magnets or a clamp such as the one described above or any other suitable additional mechanism or component may be provided, to physically hold the test accessory in place whilst (and after) it goes through the step of detaching the hook and latch (or the other components) that mirror the mechanical attachments on the test accessory that is being (or has just been) emulated. The test accessory can have its settings changed, under computer control that may be automatic, between one ‘detachment’ and a subsequent ‘attachment’, whilst being held physically in place by the additional mechanism or component, in order to make the radiotherapy device perceive that the functional accessory had been changed, and thus to change its responses accordingly, whilst in reality the same test accessory has remained in place throughout.

In some arrangements, it may be possible to automate an arm or other mechanism, under computer control, to physically remove the test accessory from the radiotherapy device, between successive tests. That is; the arm or other mechanism may hold the test accessory, securely, physically remote from the LINAC, whilst its settings are being changed in order to change the identity of the real accessory that is being emulated, ready for a next attachment and test. However; in many cases it may be more time efficient to keep the test accessory physically in place, in contact with the radiotherapy device, between tests.

The possibility of providing autonomous or semi-autonomous testing provides scope for testing radiotherapy devices at times at which, until now, it has been impractical to test them—for example, overnight. This in turn will make radiotherapy devices more available for providing radiotherapy treatment at times that are generally suitable to both patients and clinicians.

The provision of a test accessory that can mimic or emulate a plurality of real accessories, for test purposes, cuts down on the time, inconvenience and difficult manual labour that has, to date, been involved in accessory testing for radiotherapy systems. Therefore the testing becomes more efficient, without loss of accuracy. Because the test accessory does not need all the physical components that a functional accessory (such as an electron applicator) needs, the test accessory can be more lightweight and more portable than functional accessories typically are. This provides scope for a wider variety of users being able to run the testing, and reduces the risk of user injury or fatigue, during the testing process, because less heavy lifting is involved than has been the case for conventional accessory testing, to date.

It will be appreciated that, in some cases, it may not be permissible for tests using a test accessory to replace all instances of functional accessory testing, for a radiotherapy system. That is; it may be deemed appropriate to conduct tests of a radiotherapy device's responses to its ‘real’ functional accessories, as opposed to its responses to a test accessory, at least periodically. Nonetheless, the test accessory can still be used to replace at least some of the current testing of ‘real’ accessories, thereby significantly improving the overall testing regime for a radiotherapy system.

In some cases, it may be possible for a radiotherapy device to issue prompts to indicate when testing is required. For example, it may indicate when a test using the test accessory is appropriate and when a more conventional ‘real’ accessory test should be conducted.

It will further be appreciated that the particular arrangement of a test accessory, as described herein, is just one example of how the principles described herein could be realised. The example test accessory arrangement described herein is configured to emulate the ‘real’ functional electron applicator shown in FIGS. 1 and 2. Other types of ‘real’ functional accessories exist, which have different respective sizes, shapes and features. Thus, test accessories may be provided that emulate those other functional accessories, wherein those test accessories may have appropriate sizes, shapes and features, in order for a radiotherapy device to perceive their presence as being the presence of the ‘real’ accessory or accessories that they are configured to emulate, for test purposes.

Although the particular example described herein has focused on imitating electron applicators, a test accessory may also (or instead) emulate other accessory types, such as shadow trays.

For example, the test accessory 300 shown in FIGS. 3 and 4 may be used to emulate shadow trays. Again, it has been recognised herein that the test accessory 300 does not actually need to comprise lead blocks of a particular configuration, in order to emulate a shadow tray of that configuration for testing the LINAC's responses to the attachment of that shadow tray. What matters instead, in this example, is that the engagement between the test accessory and the LINAC match the engagement between the shadow tray and the LINAC; and the type of signals that are usually conveyed to the LINAC to identify a particular shadow tray configuration are conveyed by (or on behalf of) the test accessory 300. As the skilled reader will know, it is typical for a shadow tray to have a multi-bit binary code, which is transmitted to a radiotherapy device when the shadow tray is attached thereto, to identify the shadow tray to the radiotherapy device. The test accessory 300 may be configured to send such a multi-bit binary code, to identify itself as a particular shadow tray, for test purposes.

When designing a test accessory, and/or when determining whether the use of a test accessory is appropriate for emulating one or more real accessories for test purposes, it may be possible to apply a set of considerations and questions, to guide the design/determination process. Those questions may, for example, comprise some or all of the following:
What is being tested? Is it the accessory itself (in which case, using a test accessory may not be appropriate) or is it another aspect of the radiotherapy system?
If another aspect of the radiotherapy system is being tested, is the full functionality of the accessory required for the test? (If the answer is yes, using a test accessory may not be appropriate or worthwhile.)
If the full functionality of the accessory is not required, consider each component or group of components of the accessory that is to be emulated, and consider:
   Is the end result that the component contributes to (e.g. its effect on the radiation beam) required, for the test?
   If the end result is required, the component may have to be included in the test accessory, subject to the additional considerations herebelow.
   If the end result is not required, is the physical presence of that component nonetheless required, for example for inter-engagement between the test accessory and the radiotherapy device? (If it is, the component may have to be included in the test accessory.)
   If the end result is not required, is the actual physical presence of the component required, or would a signal indicating the presence of that component be sufficient (e.g. for the aperture of a particular size and shape, in the example shown in FIGS. 3 and 4 herein)?
   If neither the end result nor physical presence nor a signal indicating physical presence of a component is required, it may be deemed appropriate to omit the component.
   If neither function nor actual physical presence is required, but a signal indicating the physical presence of a component is required, it may be deemed appropriate to emulate the component.
   If the physical presence of the component is required, does it need to be provided by exactly the same component or components? Is there a way of streamlining it or simplifying it? Are all physical aspects of the component needed? Can any other improvements be made (e.g. to enable remote or automated control of the component)?

Thus, it can be seen that one can consider providing a test accessory to emulate any ‘real’ functional accessory for a radiotherapy device. The set of considerations may also include considering how many different real accessories a single test accessory can emulate, but in practice it is likely that any set of real accessories for a radiotherapy device share several physical features that could be replicated in a test accessory, or in a kit or set of components that may work together to act as a test accessory.

The example above mentions a ‘single’ test accessory to replicate multiple real accessories, but it may be possible to provide more than one test accessory, for a radiotherapy system. Moreover, the test accessory in the example given herein is a single piece accessory, but that need not be the case. Multiple components could be provided, which work in conjunction with one another in order to emulate the presence of one or more ‘real’ accessories, as perceived by the corresponding radiotherapy device.

The term ‘test accessory’ or ‘accessory’ as used herein may refer to the physical entity that fits to a radiotherapy device, or it may refer to the combination of that entity with one or more controllers or processors, which may be used to control the operation or configuration of that entity.

It may be possible to provide a set of parts that can be configured in different ways, to provide test accessories of different sizes or shapes, for example to fit to different respective types of radiotherapy devices, and/or for carrying out different respective types of test.

The same test accessory may be useable for multiple different radiotherapy devices.

The terms used in the present description such as and so on are intended to be illustrative and not to be limiting. For example, the term ‘test accessory’ may be replaced with another appropriate term such as, but not limited to: ‘universal accessory’, ‘accessory’, ‘attachment’, ‘fitting’, or ‘device’.

All terms regarding direction or orientation such as ‘top’, ‘bottom’, ‘underside’, ‘topside’, ‘lateral’, ‘towards’, ‘away’, ‘left’, ‘right’, ‘up’, ‘down’ and so on are intended to be illustrative, particularly in relation to understanding the accompanying figures, and not to be limiting.

It is to be understood that the above description is intended to be illustrative, and not restrictive. Many other implementations will be apparent to those of skill in the art upon reading and understanding the above description. Although the present disclosure has been described with reference to specific example implementations, it will be recognised that the disclosure is not limited to the implementations described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. An accessory for a radiotherapy device comprising:
a first electrical connector for forming an electrical connection to the radiotherapy device; and
a signal emitter configured to emit a signal such that the accessory emulates a connection of a second accessory to the radiotherapy device, the second accessory comprising:
a beam control apparatus.

2. The accessory of claim 1, further comprising:
a first mechanical connector configurable to form a mechanical connection to the radiotherapy device.

3. The accessory of claim 1, wherein the beam control apparatus comprised within the second accessory comprises any of: an aperture, a collimator, a beam deflector, a beam absorber, or a block.

4. The accessory of claim 2, wherein the first mechanical connector is configured for remotely-controlled actuation, in order to mechanically connect or disconnect the accessory to or from the radiotherapy device.

5. The accessory of claim 1, wherein the first electrical connector is configured for remote-control, in order to electrically connect or disconnect the accessory to or from the radiotherapy device.

6. A radiotherapy system comprising:
a radiotherapy device; and
an accessory; the accessory comprising:
a first electrical connector configurable to form an electrical connection to the radiotherapy device; and
a signal emitter configurable to emit a signal such that the accessory emulates a connection of a second accessory to the radiotherapy device.

7. A radiotherapy system as claimed in claim 6, and further comprising h second accessory, wherein the second accessory comprises:
a second electrical connector for forming an electrical connection to the radiotherapy device; and
a beam control apparatus.

8. The radiotherapy system of claim 7, wherein the second accessory comprises a second mechanical connector for forming a mechanical connection to the radiotherapy device.

9. The radiotherapy system of claim 8, wherein first accessory comprises a first mechanical connector, wherein said first mechanical connector is of a same type as the second mechanical connector.

10. The radiotherapy system of claim 7, wherein the first electrical connector is of a same type as the second electrical connector.

11. A method of testing a radiotherapy system comprising a radiotherapy device and a first accessory, wherein the first accessory comprises:
a first electrical connector for forming an electrical connection to the radiotherapy device; and
a signal emitter configured to emit a signal such that the first accessory emulates a connection of a second accessory, comprising a beam control apparatus, to the radiotherapy device;
the method comprising:
connecting the first electrical connector of the first accessory to the radiotherapy device;
controlling the signal emitter to emit a signal in order to emulate the connection of the second accessory to the radiotherapy device; and
testing a first operation of the radiotherapy device with the first electrical connector and the emulated connection of the second accessory connected to the radiotherapy device.

12. The method of claim 11, further comprising:
connecting a first mechanical connector of the first accessory to the radiotherapy device before testing the first operation of the radiotherapy device.

13. The method of claim 11, comprising, after testing the first operation of the radiotherapy device:
updating at least one property of the second accessory; and
subsequently testing a second operation of the radiotherapy device with the first electrical connector and the emulated connection of the updated second accessory connected to the radiotherapy device.

14. The method of claim 13 further comprising, after testing the first operation of the radiotherapy device, and before testing a second operation of the radiotherapy device:
disconnecting the first electrical connector from the radiotherapy device;
updating at least one property of the second accessory; and
reconnecting the first electrical connector to the radiotherapy device.

15. The method of claim 14, wherein at least one of the disconnection or the reconnection of the first electrical connector, from or to the radiotherapy device is remotely controlled.

16. The method of claim 14, wherein the first accessory further comprises:
a first mechanical connector for forming a mechanical connection to the radiotherapy device;
and wherein the method further comprises:
connecting the first mechanical connector to the radiotherapy device, before testing the first operation of the radiotherapy device;
disconnecting the first mechanical connector from the radiotherapy device after testing the first operation of the radiotherapy device, and before testing the second operation of the radiotherapy device;
updating at least one property of the second accessory; and
reconnecting the first mechanical connector to the radiotherapy device.

17. The method of any of claim 11 wherein the method is a computer-implemented method.

18. (canceled)

19. A non-transitory computer readable medium with instructions stored thereon that, when executed by a processor of a computer, cause the processor to perform operations, the operations comprising:
connecting a first electrical connector of a first accessory to a radiotherapy device;
controlling a signal emitter of the first accessory to emit a signal to emulate the connection of a second accessory to the radiotherapy device; and
testing a first operation of the radiotherapy device with the first electrical connector and the emulated connection of the second accessory connected to the radiotherapy device.

20. The non-transitory computer readable medium of claim 19, wherein the first accessory comprises:
a first mechanical connector for forming a mechanical connection to the radiotherapy device.

21. The non-transitory computer readable medium of claim 20, the operations further comprising:
connecting the first mechanical connector to the radiotherapy device prior to testing the first operation of the radiotherapy device;
updating at least one property of the second accessory;
controlling the signal emitter of the first accessory to emit a signal to emulate the connection of the updated second accessory to the radiotherapy device; and
testing a second operation of the radiotherapy device with the first electrical connector and the emulated connection of the updated second accessory connected to the radiotherapy device.
